(19) United States
(12) Patent Application Publication
(10) Pub. No.: US 2005/0222550 A1
(43) Pub. Date: Oct. 6, 2005

(54) Diaper-containing package

(75) Inventors: Koichiro Mitsui, Mitoyo-gun (JP); Hironao Minato, Mitoyo-gun (JP); Tomoyo Kawaguchi, Mitoyo-gun (JP)

Correspondence Address: LOWE HAUPTMAN GILMAN AND BERNER, LLP, 1700 DIAGONAL ROAD, SUITE 300 /310, ALEXANDRIA, VA, 22314, US

(73) Assignee: UNI-CHARM CORPORATION, Shikokuchuo-shi (JP)

(21) Appl. No.: 11/098,376

(22) Filed: Apr. 5, 2005

(30) Foreign Application Priority Data
Apr. 6, 2004 (JP) 2004-111927

(51) Int. Cl. A61F 13/15

(52) U.S. Cl. 604/385.201; 206/440; 206/570

(57) ABSTRACT

The diaper-containing package comprises a plurality of a diaper which are folded transversely inward at the opposite side portions and is folded longitudinally at the crotch region, and a bag for packaging the diapers therein. The bag forms top and bottom walls and front and back walls and opposite side walls each extending between the top and bottom walls. In the diaper, slip-resistant strips are attached to the front and back end edges and opposite side portions and these slip-resistant strips are in contact with and releasably engaged with each other. In the package, the plurality of diapers are arranged between the opposite side walls, while overlapping the waist regions each other and crotch regions each other; the front and back end edges of these diapers face to the top wall, and the crotch regions of these diapers face to the bottom wall.

### BACKGROUND OF THE INVENTION

The present invention relates to diaper-containing packages comprising a plurality of disposable diapers and a bag for packaging therein the plurality of diapers.

There are known diaper-containing packages which comprise a plurality of open type disposable diapers each having a liquid absorbent core between a liquid pervious topsheet and a liquid impervious backsheet and a bag for packaging therein the plurality of diapers. The bag forms top and bottom walls which are spaced opposite to each other in the perpendicular direction, and front and back walls and opposite side walls extending between the top and bottom walls. The plurality of diapers are contained compactly in a space surrounded by these walls. Such a diaper-containing package is disclosed, for example, in Published Japanese translation of PCT international publication for patent application No. 1999-501275. Each of the diapers has front and back waist regions, a crotch region extending therebetween, front and back end edges extending in the transverse direction and opposite side edges extending in the longitudinal direction. The diaper has the opposite side portions folded inward with the topsheet inside and then is folded inside in the crotch region with the folded halves of the topsheet contacted with each other. In the folded diaper, the front and back end edges as well as the opposite side portions are in contact with each other.

The above-described diaper-containing package includes the bag made of a film in the form of a hexahedron, wherein the top and bottom walls, front and back walls, and opposite side walls cross each other at generally right angles, and the package has a rectangular cuboid longer in the perpendicular direction. In the package, these diapers are arranged between the opposite side walls while they are overlapped with each other both at the waist regions and the crotch region. The plurality of diapers form two rows extending between the opposite side walls. These two rows are stacked perpendicularly in the overlapped state. In the upper row, the crotch region of each of these diapers is adjacent to the top wall and the front and back end edges face to the bottom wall, while in the lower row, the crotch region is adjacent to the bottom wall and the front and back end edges face to the top wall. In each of the diapers at both ends of these rows, the waist region and crotch region are adjacent to the side walls.

In the package disclosed in the above-described diaper-containing package, the topsheet and backsheet constituting each of the diapers have a low friction so that it is difficult to maintain the folded shape of each diaper due to easy misalignment of the front and back waist regions of the folded diaper which are in contact with each other. An external force applied causes the shape loss of the folded diapers and the diapers partially distort the bag. This leads to a shape loss of the package, making it impossible to maintain the cuboid shape of the package. When plural packages losing their shape are stacked one after another, they are instable and collapse improperly.

### SUMMARY OF THE INVENTION

An object of the present invention is to provide a diaper-containing package which can maintain folded shape of diapers and are free of the shape loss.

According to the present invention, there is provided a diaper-containing package which comprises a plurality of open type disposable diapers each having a liquid pervious topsheet, a liquid impervious backsheet and a liquid absorbent core extends therebetween, and bag made of a flexible film packaging therein the plurality of diapers, the diapers each having front and back waist regions, a crotch region extending therebetween, front and back end edges extending in transverse direction, and opposite side portions extending in longitudinal direction; the bag forming top and bottom walls which are spaced opposite to each other in a perpendicular direction, front and back walls opposite to each other and extending between the top and bottom walls, and opposite side walls opposite to each other and extending between the top and bottom walls; and the plurality of diapers being compactly contained forming a row in a space surrounded by the walls with the opposite side portions of each diaper folded inward so as to be sandwiched between the front and back waist regions and with each diaper folded in two inward along the crotch region so as for the topsheet to oppose to each other.

The diaper-containing package according to the present invention further comprises a slip-resistant strip having a higher friction force than that of the topsheet and backsheet being attached to at least a part of the opposite side portions in the front waist region of each diaper and at least a part of the opposite side portions in the back waist region of each diaper; and each of the front back waist regions being overlapped at the opposite side portions thereof so that slip-resistant strips attached to the opposite side portions respectively are releasably engaged with each other; and said plurality of diapers being arranged forming a row between the opposite side walls of the bag, diapers at the waist regions and at the crotch region; and the front and back end edges face to either one of the top and bottom walls while the crotch region faces to the other one of the top and bottom walls.

The present invention includes the following embodiments.

(1) According to one embodiment of the present invention, the slip-resistant strip is attached to at least a part of the front end edge extending in the front waist region of the diaper and at least a part of the back end edge extending in the back waist region; and in the folded diaper, the front and back end edges are overlapped each other and the slip-resistant strips attached to the front and back end edges are in contact with and releasably engaged with each other.

(2) According to another embodiment of the present invention, the slip-resistant strips are each made of a composite nonwoven fabric obtained by laminating a first nonwoven fabric made of fibers of a thermoplastic synthetic resin having rubber elasticity with a second nonwoven fabric made of fibers of a polyolefin thermoplastic synthetic resin; and in the slip-resistant strips, the first nonwoven fabrics are in contact with and releasably engaged with each other.

(3) According to a further embodiment of the present invention, the respective first nonwoven fabrics constituting the slip-resistant strips are engaged at a releasable engagement strength ranging from 0.5 to 1.5 N/25 mm.

(4) According to a still further embodiment of the present invention, the first nonwoven fabric constituting the slip-resistant strip has a dynamic friction coefficient ranging from 0.5 to 1.5.

(5) According to yet further embodiment of the present invention, in the package, at least two of the rows constituted of a plurality of the diapers are arranged perpendicularly in the stacked form; in the rows stacked each other, the front and back end edges of the diaper face to either one of the top and bottom walls, and the crotch region of the diaper faces to the other one of the top and bottom walls.

According to the diaper-containing package of the present invention, the slip-resistant strip having a higher friction force than that of the topsheet and backsheet is attached to at least a part of the opposite side portions extending in the front waist region of the diaper, and at least a part of the opposite side portions extending in its back waist region and these slip-resistant strips attached to the two end portions in the front and back waist regions are in contact with and releasably engaged with each other so that the misalignment of the front and back waist regions of the folded diaper which are in contact with each other can be prevented by the slip-resistant strip. Even if an external force is applied to the package, the folded diaper does not lose its shape. This package can maintain the folded shape of the diapers by using these slip-resistant strips, and the diapers do not partially distort the bag, whereby the package can be prevented from losing its shape. The plurality of packages free from such a shape loss are stably stacked one after another in the perpendicular direction and do not collapse improperly.

In the package in which the slip-resistant sheet is attached to at least a part of the front end edge extending in the front waist region and at least a part of the back end edge extending in the back waist region, and these slip-resistant strips attached to these front and back end edges are in contact with and releasably engaged with each other, misalignment not only at the opposite side portions of the diaper but also at the front and back end edges can be prevented by these slip-resistant sheets, whereby the misalignment in the front and back waist regions of the folded diaper which are in contact with each other can be prevented reliably. Thus, by using the slip-resistant strips, the folded shape of the diaper can be maintained while reliably preventing the package from losing its shape.

In the package of the diapers wherein the slip-resistant strips are each made of a composite nonwoven fabric obtained by laminating a first nonwoven fabric made of the fibers of a thermoplastic synthetic resin having a rubber elasticity with a second nonwoven fabric made of the fibers of a polyolefin thermoplastic synthetic resin and the first nonwoven fabrics are in contact with and releasably engaged with each other, the high friction force of the first nonwoven fabric can reliably prevent the misalignment of the front and back waist regions of the folded diaper which are in contact with each other and the shape loss of the package while maintaining the folded shape of the diaper.

In the package wherein the first nonwoven fabrics constituting the slip-resistant strips are engaged with each other at a releasable engagement strength ranging from 0.5 to 1.5 N/25 mm, even an external force applied to the package does not cause improper peeling of the first nonwoven fabrics which have been in contact with each other. By using the first nonwoven fabric, the misalignment between the front and back waist regions of the folded diaper which are in contact with each other can be prevented reliably and in addition, the distortion of the package can be prevented reliably while maintaining the folded shape of the diaper.

In the package wherein the first nonwoven fabric constituting the slip-resistant strip has a dynamic friction coefficient ranging from 0.5 to 1.5, the first nonwoven fabric has an adequate friction force. By using the first nonwoven fabric, the misalignment between the front and back waist regions of the folded diaper which are in contact with each other can be prevented reliably and in addition, the distortion of the package can be prevented reliably while maintaining the folded shape of the diaper.

In the diaper-containing package having at least two rows arranged in the perpendicular direction in the stacked form, even friction between the diapers constituting these rows causes neither shape loss of the folded diapers in these rows nor shape loss of the package, because misalignment between the front and back waist regions of the folded diaper which are in contact with each other is prevented by the slip-resistant strip.

### BRIEF DESCRIPTION OF THE DRAWINGS

FIG. 1 is a perspective view of a diaper-containing package according to one embodiment;

FIG. 2 is a perspective view of the diaper taken out from a bag;

FIG. 3 is a perspective view of the diaper which is longitudinally unfolded, at the front and back waist regions thereof, from the state of FIG. 2;

FIG. 4 is a partially cutaway perspective view of the diaper which is transversely unfolded, at the opposite side portions thereof, from the state of FIG. 3;

FIG. 5 is a schematic view illustrating the measuring method of a dynamic friction coefficient;

FIG. 6 is a perspective view illustrating a diaper-containing package according to another embodiment;

FIG. 7 is a perspective view of the diaper taken out from a bag;

FIG. 8 is a perspective view of the diaper which is longitudinally unfolded, at the front and back waist regions thereof, from the state of FIG. 7; and

FIG. 9 is a partially cutaway perspective view of the diaper which is transversely unfolded, at the opposite side portions thereof, from the state of FIG. 8.

### DETAILED DESCRIPTION OF THE PREFERRED EMBODIMENTS

The diaper-containing package according to the present invention will next be described specifically referring to the accompanying drawings.

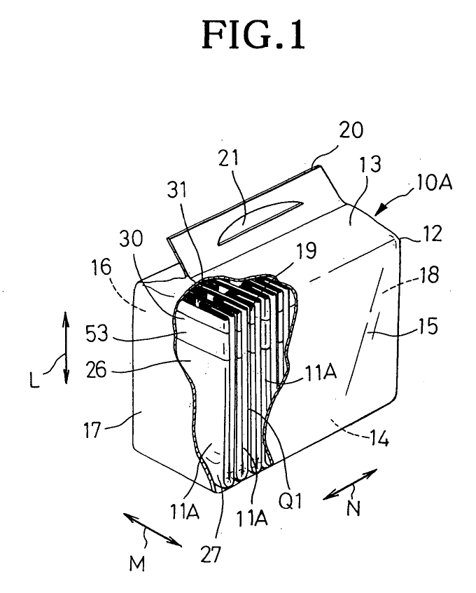

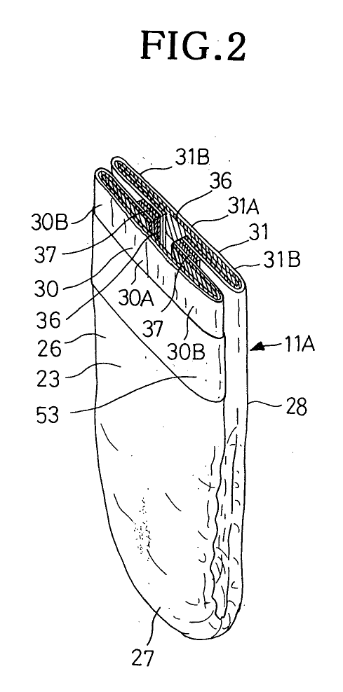

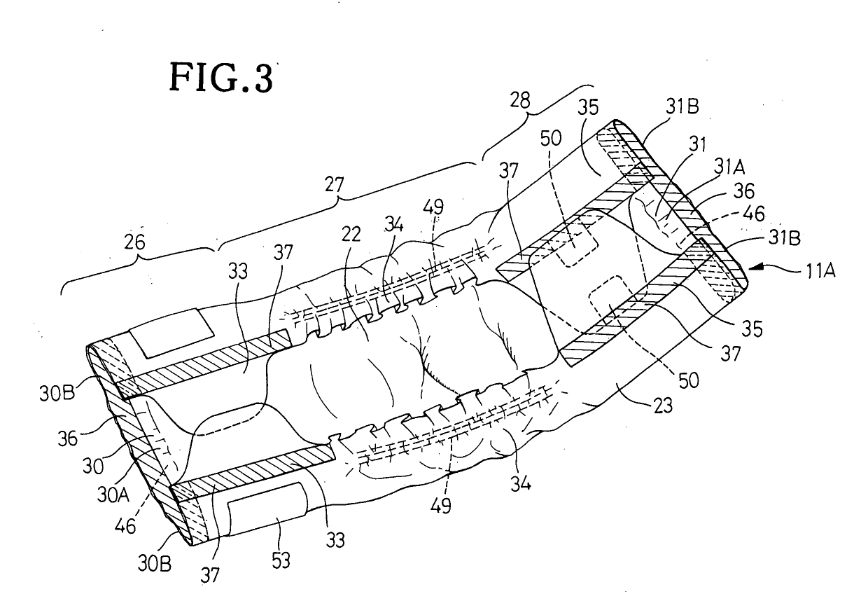

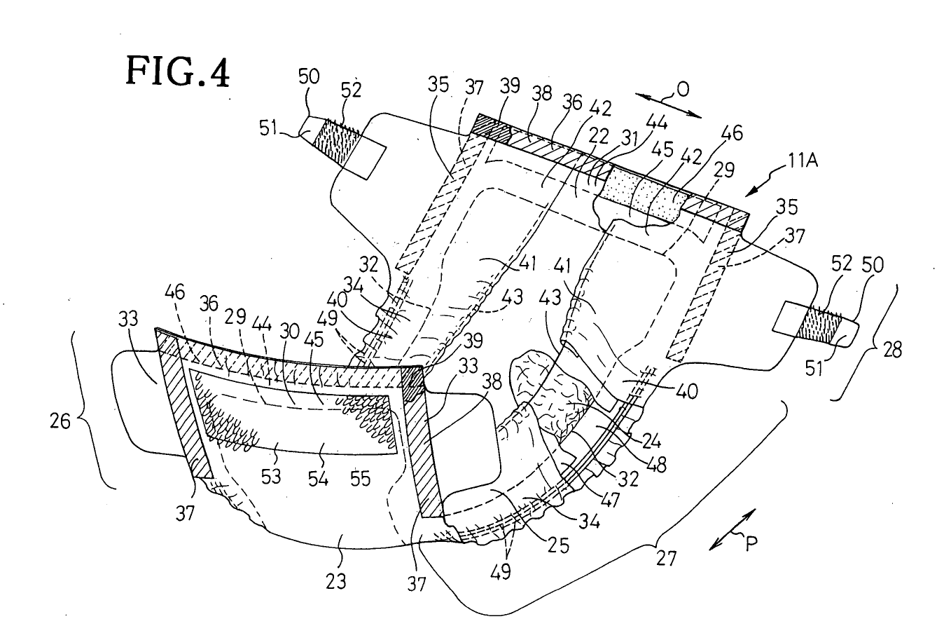

FIGS. 1 and 2 are a perspective view of a diaper-containing package 10A shown as one example, and a perspective view of a diaper 11A taken out from the package 10A, respectively. FIGS. 3 and 4 are a perspective view of the diaper 11A in which front and back waist regions 26, 28 are unfolded from the state of FIG. 2 in the longitudinal direction and a partially broken perspective view of the diaper 11A in which opposite side portions 33, 34, 35 are unfolded from the state of FIG. 3 in the transverse direction, respectively. In FIG. 1, a top wall 13, front wall 15 and side wall 17 are illustrated in the partially broken form. In FIG. 1, the perpendicular direction is indicated by an arrow L, the front-to-back direction is indicated by an arrow M and the transverse direction is indicated by N. In FIG. 4, the longitudinal direction is indicated by an arrow P, while the transverse direction is indicated by an arrow O. In these drawings, the term “inner surface” of the topsheet and backsheet 22, 23 or leak-barrier sheet 25 means a surface facing to the core 24, while the term “outer surface” of these sheets 22, 23, 25 means a face not facing to the core 24.

The diaper-containing package 10A comprises a plurality of disposable diapers 11A and a bag 12 made of a flexible film wrapping therein the plurality of diapers 11A. The bag 12 has top and bottom walls 13, 14 spaced opposite to each other in the perpendicular direction, front and back walls 15, 16 extending in the perpendicular direction contiguously to the top and bottom walls 13, 14, and opposite side walls 17, 18 extending in the perpendicular direction contiguously to the top and bottom walls 13, 14. The front and back walls 15, 16 are spaced opposite to each other in the front-to-back direction and extend between the top and bottom walls 13, 14. The opposite side walls 17, 18 are spaced opposite to each other in the transverse direction and extend between the top and bottom walls 13, 14. The package 10A is in the form of a hexahedron with the top and bottom walls 13, 14, the front and back walls 15, 16 and opposite side walls 17, 18 of the bag 12 crossing at substantially right angles. Its steric shape is a cuboid. In the bag 12, a space 19 encompassed by the walls 13, 14, 15, 16, 17, 18 formed to contain the plurality of diapers 11A therein compactly. At the top wall 13, a handle 20 is formed for carrying the package 10A. The handle 20 is made of a part of the bag 12 and extends upward from the top wall 13. The handle 20 has at the transversely center region thereof, an opening 21 permitting insertion of the hand of a carrier and extending in the transverse direction.

The diaper 11A comprises a liquid pervious topsheet 22 facing the wearer, a liquid impervious backsheet 23 facing the wearer's garment, an absorbent core 24 disposed between the topsheet and backsheet 22, 23 and bonded to at least one of the inner surfaces of these sheets 22, 23, and a pair of liquid impervious leak-barrier sheets 25 spaced opposite to each other in the transverse direction and extending in the longitudinal direction. The diaper 11A has, in the longitudinal direction thereof, a front waist region 26, a back waist region 28 and a crotch region 27 extending between these waist regions 26, 28. The diaper 11A has front and back end edges 30, 31 extending transversely outward of the two end edges 29 of the core 24, and opposite side portions 33, 34, 35 extending longitudinally outward of the opposite side edges 32 of the core 24. The diaper 11A has a generally hourglass planar shape and is an open type diaper in which the front and back waist regions 26, 28 are connected for wearing (refer to FIG. 4).

The diaper 11A is provided to the frond end edge 30 and the back end edge 31 with rectangular slip-resistant strips 36 extending in the transverse direction. The diaper 11A is also provided to a part of the opposite side portions 33 in the front waist region 26 and a part of the opposite side portions 35 in the back waist region 28 with rectangular slip-resistant strips 37 extending in the longitudinal direction. These slip-resistant strips 36, 37 have a higher friction force than that of the topsheet and backsheet 22, 23 or leak-barrier sheet 25. The slip-resistant strips 36, 37 are each made of a composite nonwoven fabric obtained by laminating a first nonwoven fabric 38 of elastic fibers made from a thermoplastic synthetic resin having a rubber elasticity with a second nonwoven fabric 39 of non-elastic fibers made from a thermoplastic synthetic resin. The elastic fibers are entangled and joined together at their crossings by thermal fusion. The non-elastic fibers are entangled together and joined together at their crossings by thermal fusion. The elastic fibers and non-elastic fibers are entangled and joined together at their crossing thereof by thermal fusion. When a certain pressure is applied to the sheets 36, 37 while bringing the first nonwoven fabrics 38 into contact with each other, the first nonwoven fabrics 38 can be releasably engaged with each other.

The slip-resistant strip 36 has the first nonwoven fabric 38 disposed on the side contacted with the wearer's skin and the second nonwoven fabric 39 disposed on the side away from while being bonded to the outer surface of the topsheet 22 and the outer surface of the leak-barrier sheet 25 by the second nonwoven fabric 39. In the slip-resistant strip 36, the first nonwoven fabric 38 is exposed on the side contacted with the wearer's skin. The slip-resistant strip 37 has the first nonwoven fabric 38 disposed on the side away from the wearer's skin and the second nonwoven fabric 39 disposed on the side contacted with the wearer's skin, while being bonded to the outer surface of the backsheet 23 by the second nonwoven fabric 39. In the slip-resistant strip 37, the first nonwoven fabric 38 is exposed on the side away from the skin.

The composite nonwoven fabric forming the slip-resistant strips 36, 37 may be produced by the known meltblown process or spunbond process. As a thermoplastic synthetic resin constituting the elastic fibers, any one of styrene block copolymers, polyurethane block copolymers, polyester block copolymers, polyamide block copolymers, and copolymer blends may be used. Examples of the styrene block copolymers include styrene-butadiene-styrene (S-B-S) and styrene-ethylene butadiene-styrene (S-EB-S). Examples of the copolymer blend include styrene-ethylene butadiene-styrene/polypropylene (S-EB-S/PP) and polypropylene/ethylene-propylene (PP/E-P). As the thermoplastic synthetic resin constituting the non-elastic fibers include polyolefin resins. As the polyolefin resins, any one of polyamide, polyester, polyethylene and polypropylene resins may be used.

The diapers 11A packed in the bag 12 have the opposite side portions 33, 34, 35 thereof folded inward into two along the longitudinally center portion in the crotch region 27 so as to bring opposite surfaces of the folded topsheet 22 into contact with each other (refer to FIG. 2). In the folded diaper 11A, the front and back waist regions 26, 28 are overlapped, the folded portions of the crotch region 27 are overlapped, the front and end edge portions 30, 31 are overlapped, and the folded portions of each of the opposite side portions 33, 34, 35 are overlapped. The front end edge 30 is overlapped at the transversely opposite side portions 30B except the transversely central portion 30A, while the back end edge 31 is overlapped at the transversely opposite side portions 31B except the transversely central portion 31A. The front end edge 30 and the back end edge 31 are overlapped at the transversely central portions 30A, 31A of the end edges 30, 31. At the opposite side portions 30B of the front end edge 30, the first nonwoven fabrics 38 forming the slip-resistant strip 36 are releasably engaged with each other. At the opposite side portions 31B of the back end edge 31, the first nonwoven fabrics 38 forming the slip-resistant strip 36 themselves are releasably engaged with each other. At the central portions 30A, 31A of the front and back end edges 30, 31, the first nonwoven fabrics 38 are releasably engaged with each other. At the opposite side portions 33, 35 of the front and end waist regions 26, 28, the first nonwoven fabrics 38 constituting the slip-resistant strip 36 are releasably engaged with each other.

A plurality of the diapers 11A are arranged between the opposite side walls 17, 18 while they are overlapped each other at the waist regions 26, 28 and at the crotch regions 27. A group of these diapers 11A forms a row Q1 extending between the opposite side walls 17, 18. In the row Q1, the front and back end edges 30, 31 of the diapers 11A are laid in a row between the opposite side walls 17, 18 and face to the top wall 13, and the front and back end edges 30, 31 are in contact with the top wall 13, while the crotch regions 27 are laid in a row between the opposite side walls 17, 18 and face to the bottom wall 14, and the crotch regions 27 are in contact with the bottom wall 14. At both sides of the row Q1, the waist regions 26, 28 and the crotch region 27 of the diaper 11A are in contact with the opposite side walls 17, 18. The row Q1 is sandwiched between the top and bottom walls 13, 14 and is therefore perpendicularly compacted; is sandwiched between the front and back walls 15, 16 and is therefore compacted in the front-back direction; and sandwiched between the opposite side walls 17, 18 and is compacted in the horizontal direction.

Referring to FIG. 4, the other constitution of the diaper 11A will be described. The leak-barrier sheet 25 is disposed at the opposite side portions 33, 34, 35 of the diaper 11A. The leak-barrier sheet 25 has a fixed side portion 40 existing at the opposite side portions 33, 34, 35 and extending in the longitudinal direction, a free portion 41 extending in the longitudinal direction parallel to the fixed side portion 40 and biasing to rise up above the topsheet 22, and fixed opposite end portions 42 existing at the front and back end edges 30, 31 and laid transversely inward of the diaper 11A. To the upper part of the free portion 41, a stretchable elastic member 43 is contractibly secured to the free portion 41 while being extended by a predetermined ratio in the longitudinal direction. When the diaper 11A is bent with the topsheet 22 inside, the contraction of the elastic member 43 occurs, the free portion 41 rises up above the topsheet 22 and the free portion 41 forms a barrier for exudates.

The front and back end edges 30, 31 are comprised of the end portions 44, 45 of the topsheet and backsheet 22, 23 extending longitudinally outward from the end edge 29 of the core 24, and the fixed opposite end portions 42 of the leak-barrier sheet 25. At the front and back end portions 30, 31, the end portions 44, 45 of the topsheet and backsheet 22, 23 are superposed with the end portion 42 of the leak-barrier sheet 25, the inner surfaces of the topsheet and backsheet 22, 23 are bonded each other, and the inner and outer surfaces of the topsheet and backsheet 22, 23 are bonded to the inner surface of the leak-barrier sheet 25. To the front and back end edges 30, 31, a strip-like waist elastic member 46 extending in the transverse direction is contractibly secured. The waist elastic member 46 is inserted between the end portion 44 of the topsheet 22 and the end portion 45 of the backsheet 23 and is bonded to the inner surfaces of these sheets 22, 23 while being extended at a predetermined ratio in the transverse direction.

The opposite side portions 33, 34, 35 are comprised of side portions 47, 48 of the topsheet and backsheet 22, 23 extending transversely outwards from the side edge 32 of the core 24 and the fixed side portion 40 of the leak-barrier sheet 25. At the opposite side portions 33, 34, 35, the side portion 47 of the top sheet 22 slightly extends transversely outwards form the side edge 32 of the core 24 from the side portion 47, the side portion 48 of the backsheet 23 and the side portion 40 of the leak-barrier sheet 25 extend transversely outwards. At the opposite side portions 33, 34, 35, the side portions 47, 48 of the topsheet and backsheet 22, 23 are overlapped with the side portion 40 of the leak-barrier sheet 25 and the inner surfaces of the topsheet and backsheet 22, 23 are bonded to each other and the inner and outer surfaces of the topsheet and backsheet 22, 23 are bonded to the inner surface of the leak-barrier sheet 25. At the opposite side portions 34, a plurality of longitudinally-extending elastic members 49 of strands for surrounding the legs of the wearer are contractibly secured. The leg-surrounding elastic members 49 are inserted between the side portion 48 of the backsheet 23 and the side portion 40 of the leak-barrier sheet 25. The members 49 are bonded to the inner surfaces of these sheets 23, 25 while being extended by a predetermined ratio in the longitudinal direction.

At each of the opposite side portions 35 of the back waist region 28, a flexible tape fastener 50 made of a fibrous nonwoven fabric is attached. At the free end portion 51 of the tape fastener 50, a hook member 52 is attached. The free end portion 51 is folded inwards in the transverse direction of the diaper 11A and is fixed detachably to the opposite side portion 35 by the hook member 52 (refer to FIG. 3). At the front waist region 26, a flexible target tape 53 for detachably fixing the free end portion 51 of the tape fastener 50 thereto is attached. The target tape 53 has a rectangular shape longer in the transverse direction and is comprised of a plastic film 54 and a loop member 55. The target tape 53 is attached to the outer surface of the backsheet 23.

For wearing the diaper 11A, it is necessary to tear the film 12, take out the diaper 11A from the bag 12, unfold the diaper 11A at the crotch region 27 to spread the front and back waist regions 26, 28, which have been in contact with each other, in the longitudinal direction, unfolding the opposite side portions 33, 34, 35 to spread them in the transverse direction. Subsequently, it is necessary to overlap the opposite side portions 35 of the back waist region 28 with the outside of the opposite side portions 33 in the front waist region 26, fixing the free end 51 of the tape fastener 50 to the target tape 53 to connect the front waist region 26 with the back waist region 28. As the front and back waist regions 26, 28 are connected with each other, the diaper 11A has a waist opening and a pair of leg-openings formed therein (not illustrated).

In this diaper-containing package 10A, slip-resistant strips 36, 37 having a higher friction force than that of the topsheet and backsheet 22, 23 or that of the leak-barrier sheet 25 are attached to the front and back end edges 30, 31 and opposite side portions 33, 35; and the first nonwoven fabrics 38 constituting the slip-resistant strips 36, 37 are in contact with and releasably engaged with each other. Therefore, the slip-resistant strips 36, 37 prevent the misalignment of the diaper 11A at the front and back end edges 30, 31 and opposite side portions 33, 35 and the misalignment of the front and back waist regions 26, 28 in contact with each other of the folded diaper 11A can be suppressed by the slip-resistant strips 36, 37. Even when an external force is applied to the package 10A, the folded diaper 11A contained in the bag 12 does not lose its shape improperly.

In the package 10A, the folded shape of the diaper 11A can be maintained by using the slip-resistant strips 36, 37, and the diapers 11A do not cause a partial distortion of the film 12, making it possible to prevent the package 10A from losing its shape. Since the package 10A is free from the shape loss, a plurality of the packages 10A can be stacked perpendicularly one after another stably and the stacked packages 10A do not collapse improperly.

The releasable engagement strength between the first nonwoven fabrics 38 constituting each of the slip-resistant strips 36, 37 ranges from 0.5 to 1.5 N/25 mm. The releasable engagement strength between the first nonwoven fabrics 38 less than 0.5 N/25 mm cannot prevent the misalignment of the front and back waist regions 26, 28 of the diaper 11A, and when an external force is applied to the package 10A the folded diaper 11A lose its shape easily to cause the distortion of the package 10A. The releasable engagement strength between the first nonwoven fabrics 38 was measured in the following manner.

(1) The folded diaper 11A was cut into a sample piece for measuring a releasable engagement strength which sample includes the first nonwoven fabrics 38 in contact with and releasably engaged with to each other. The sample was 100 mm wide and 70 mm long. The releasable engagement strength of the sample was measured using “Autograph AG-50NI” (trade name; product of Shimadzu System Solutions).

(2) The sample was pinched with the chucks of the measuring apparatus (chuck-chuck distance: 30 mm); the releasably engaged slip-resistant strips 36 or the slip-resistant strips 37 (first nonwoven fabrics 38) were pulled in a direction to separate them from each other (pulling rate; 100 mm/min); and the strength when the first nonwoven fabrics 38 were separated each other at 180° peel was measured by the measuring apparatus. The releasable engagement strength was calculated in accordance with the conversion equation: (measured value/releasably engaged width)×25 mm. The releasable engagement strength of the sample thus calculated in accordance with the equation was 0.5 to 1.5 N/25 mm. The releasable engagement strength of the sample was defined as that between the first nonwoven fabrics 38.

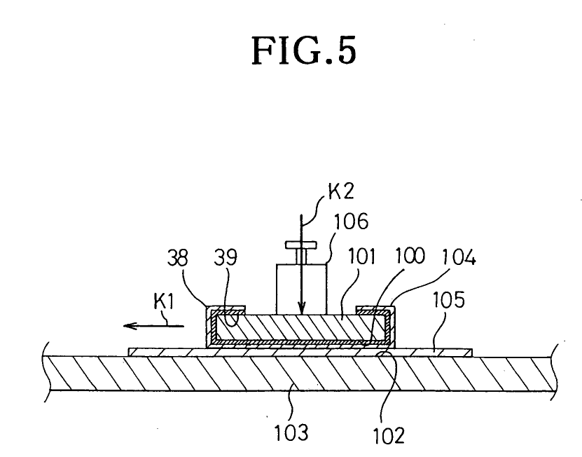

The dynamic friction coefficient of the first nonwoven fabrics 38 constituting the slip-resistant strips 36, 37 ranges from 0.1 to 1.5. The dynamic friction coefficient of the first nonwoven fabrics 38 less than 0.5 cannot prevent the misalignment of the front and back waist regions 26, 28 of the diaper 11A, and an external force applied to the package 10A easily make the folded diaper 11A lose its shape, which presumably causes the distortion of the package 10A. The dynamic friction coefficient of the first nonwoven fabrics 38 was measured in accordance with the method as described in page 3.1 of JIS P 8147. FIG. 5 is a schematic view illustrating the measuring method of the dynamic friction coefficient.

(1) For the measurement of the dynamic friction coefficient, a movable plate 101 having a smooth bottom surface 100 and a fixed plate 103 having a smooth top surface 102 were used. The bottom surface 100 of the movable plate 101 was 3 cm wide and 3 cm long. A sample 104 (composite nonwoven fabric) for measuring the dynamic friction coefficient, which was the same composite nonwoven fabric used for the slip-resistant sheets 36, 37 attached to the diaper 11A, and also a synthetic leather 105 (“SUPPLALE”, trade name; product of IDEMITSU PETROCHEMICAL CO., LTD.) were provided.

(2) The sample 104 was attached to the bottom surface 100 of the movable plate 101 while contacting the second nonwoven fabric 39 with the movable plate 101. The sample 104 was fixed to the movable plate 101 with a double-sided adhesive tape (not illustrated). The synthetic leather 105 was secured to the upper surface 102 of the fixed plate 103. The synthetic leather 105 was fixed to the fixed plate 103 with a double-sided adhesive tape (not illustrated). The movable plate 101 was laid over the fixed plate 103 to contact the first nonwoven fabric 38 with the synthetic leather 105. The bottom surface 100 of the movable plate 101 had an area of 9 cm². The whole weight of the movable plate 101 was adjusted by a sash weight 106 to apply a load of 58 g/9 cm² to the bottom surface 100.

(3) For the measurement of the dynamic friction coefficient, the movable plate 101 was used as a plummet. The movable plate 101 was moved at a rate of 10 cm/min in the direction as shown in FIG. 5 by an arrow K1. The dynamic friction coefficient was determined from the friction force required for transfering the movable plate 101 by 5 cm. More specifically, the dynamic friction coefficient was calculated based on the following equation: μ=F_{D}/Fₚ, in which μ means a dynamic friction coefficient and FD means a friction force required for transferring the movable plate 101 by 5 cm. The Fₚ is a force shown by an arrow K2 and acting in the perpendicular direction on the contact surface between the first nonwoven fabric 38 and the artificial leather 105. The dynamic friction coefficient of the sample 104 as calculated based on the above-described equation was from 0.5 to 1.5. The dynamic friction coefficient of the sample 104 was defined as that of the first nonwoven fabric 38.

The composite nonwoven fabric constituting the slip-resistant strips 36,37 has a tensile strength ranging from 30 to 70 N/inch, a basis weight ranging from 30 to 100 g/m², and a thickness ranging from 0.2 to 0.7 mm. When the composite nonwoven fabric has a basis weight less than 30 g/m² and thickness less than 0.2 mm, the strips 36, 37 have lowered tensile strength, which presumably causes breakage of the strips 36, 37 when friction between the strip 36 or the strip 37 occurs. When the basis weight and the thickness exceed 100 g/m² and 0.7 mm, respectively, the strips 36, 37 have increased rigidity and cause discomfort to the wearers in direct contact with the skin. Elastic fibers constituting the first nonwoven fabric 38 has a fineness ranging from 0.5 to 20 μm. When the elastic fibers have a fineness less than 0.5 μm, they easily cause fiber breakage, presumably causing lowering in the releasable engagement strength and the dynamic friction coefficient of the first nonwoven fabrics 38 to less than 0.5 N/25 mm and less than 0.5, respectively.

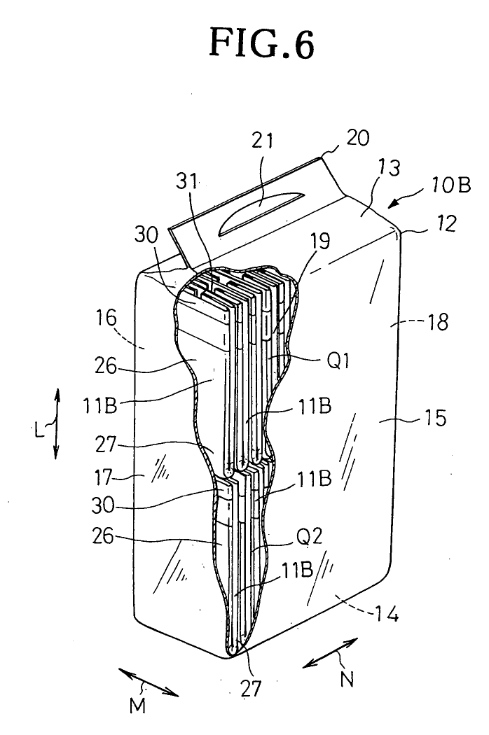

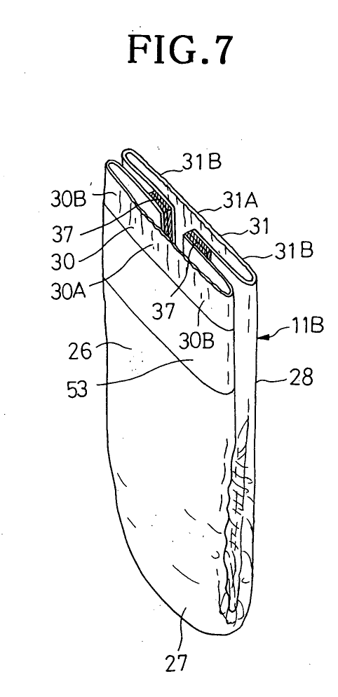

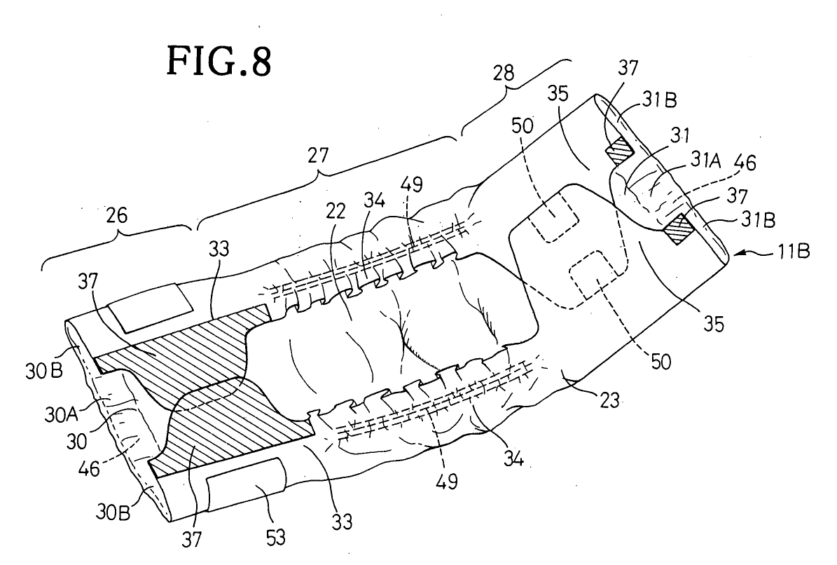

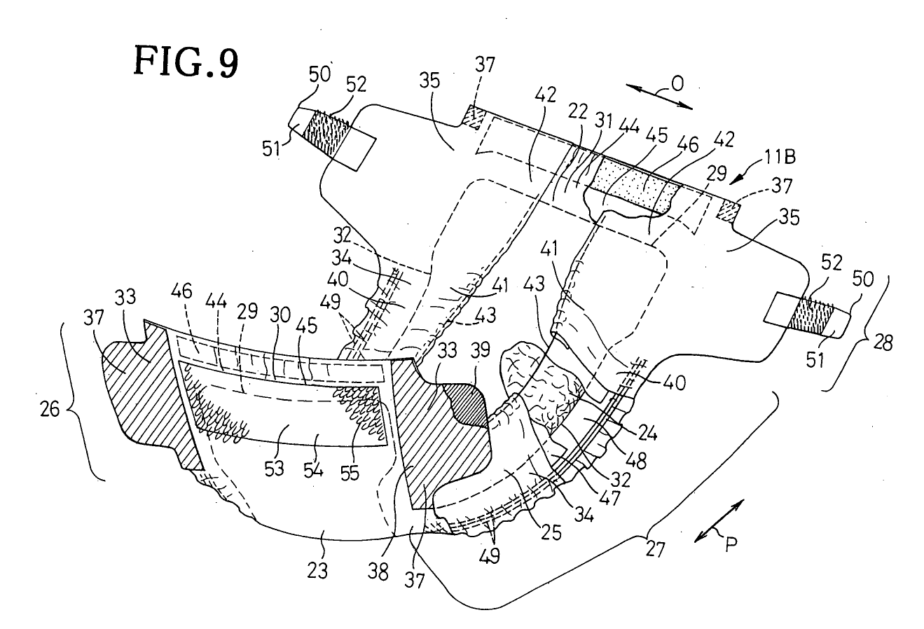

FIGS. 6 and 7 are perspective views of a diaper-containing package 10B according to another embodiment of the present invention and a diaper 11B taken out from a space 19, respectively. FIGS. 8 and 9 are a perspective view of the diaper 11B in which front and back waist regions 26, 28 are longitudinally unfolded from the state of FIG. 7 and a partially broken perspective view of the diaper 11B in which the opposite side portions 33, 34, 35 are transversely unfolded from the state of FIG. 8, respectively. FIG. 6 illustrates a part each of the top wall 12, the side wall 15 and the side wall 17 in the broken form. In FIG. 6, the perpendicular direction is indicated by an arrow L, the front-back direction is indicated by an arrow M and the horizontal direction is indicated by N. In FIG. 9, the longitudinal direction is indicated by an arrow P, while the transverse direction is indicated by an arrow O.

The package 10B for diapers 11B comprises disposable diapers 11B and a bag 12 made of a flexible film packaging therein these diapers 11B. The bag 12 constitutes top and bottom walls 13, 14 spaced opposite to each other in the perpendicular direction, front and back walls 15, 16 extending in the perpendicular direction the top and bottom walls 13, 14, and opposite side walls 17, 18 extending in the perpendicular direction contiguously to the top and bottom walls 17, 18. The package 10B is a hexahedron with the top and bottom walls 13, 14, the front and back walls 15, 16 and opposite side walls 17, 18 of the film 12 crossing at substantially right angles. Its steric shape is a cuboid longer in the perpendicular direction. In the package 10B, a space 19 encompassed by the walls 13, 14, 15, 16, 17, 18 is formed to contain a plurality of diapers 11B therein compactly. At the top wall 13, a handle 20 is formed for carrying the package 10B. The handle 20 has, at the transversely center region thereof, an opening 21 permitting insertion of a hand.

In this diaper 11B, different from that of FIG. 4, a slip-resistant sheet 37 is attached to the whole of the opposite side portions 33 of the front waist region 26 and to a part of the opposite side portions 35. The constitution of the diaper 11B except for the attached position of the slip-resistant strip 37 is similar to that of the diaper 11A as illustrated in FIGS. 2 to 4 so that the like members of diaper 11B are identified by like reference numerals and the description of the constitution of the diaper 11B other than the attached position is omitted.

At the back waist region 28, the slip-resistant strip 37 is attached to the opposite side portions 35 existing on both sides of the back edge 31. The slip-resistant strip 37 has a friction force higher than that of the topsheet and backsheet 22, 23 or the leak-barrier sheet 25. The slip-resistant strip 37 is made of a composite nonwoven fabric similar to that attached to the diaper 11A. In the slip-resistant strip 37, the first nonwoven fabric 38 exists on the side away from the wearer's skin, while the second nonwoven fabric 39 exists on the side contacted with the wearer's skin. The slip-resistant strip is bonded to the outer surface of the backsheet 23 by the second nonwoven fabric 39. In the slip-resistant strip 37, the first nonwoven fabric 38 is exposed on the side away from the wearer's skin.

The diapers 11B contained in the bag 12 are each folded, at the opposite side portions 33, 34, 35 thereof, transversely inward toward the side of the outer surface of the top sheet 22 and folded into two along the longitudinal center in the crotch region 27 so as to bring the outer surfaces of the topsheet 22 into contact with each other (refer to FIG. 7). In the folded diaper 11B, the front and back waist regions 26, 28 are overlapped each other, the folded portions of the crotch region 27 are overlapped each other, the front and back end edges 30, 31 are overlapped each other, and the folded portions of the respective opposite side portions 33, 34, 35 are overlapped each other. The front edge 30 is overlapped at the transversely opposite portions 30B thereof except the transversely center portion 30A, while the back edge 31 is overlapped at the transversely opposite portions 31B thereof except the transversely center portion 31A. The front edge 30 and the back edge 31 are overlapped each other at the transversely central portions 30A, 31A of the front and back edges 30, 31. At the opposite side portions 33, 35 in the front and back waist regions 26, 28, the first nonwoven fabrics 38 are in contact with and releasably engaged with each other.

A plurality of the diapers 11B are arranged between the opposite side walls 17, 18 while they are overlapped each other at the waist regions 26, 28 and at the crotch regions 27. A group of these diapers 11B forms two rows Q1, Q2 extending between the opposite side walls 17, 18. These rows Q1, Q2 are stacked in the perpendicular direction. In the perpendicularly upper row Q1 in the bag 12, the front and back end edges 30, 31 of the diapers 11B are laid in a row between the opposite side walls 17, 18, and face to and are in contact with the top wall 13; and the crotch regions 27 of the diapers 11B are laid in a row between the opposite side walls 17, 18 and face to the bottom wall 14. In these rows Q1 and Q2, the front and back end edges 30, 31 of the diapers 11B of the lower row Q2 are in contact with the crotch regions 27 of the diapers 11B of the upper row Q1. On both sides of the rows Q1, Q2, the waist regions 26, 28 and crotch region 27 of the diapers 11B are in contact with the opposite side walls 17, 18. The rows Q1, Q2 are sandwiched between the top and bottom walls 13, 14 and are therefore perpendicularly compacted; they are sandwiched between the front and back walls 15, 16 and are therefore compacted in the front-back direction; and they are sandwiched between the opposite side walls 17, 18 and are therefore horizontally compacted. The wearing procedure of the diaper 11B is similar to that of the diaper 10B so that the description on the procedure will be omitted.

In this diaper-containing package 10B, the slip-resistant strip 37 having a friction force higher than that of the topsheet and backsheet 22, 23 or that of the leak-barrier sheet 25 is attached to the opposite side portions 30, 31 of the front and back waist regions 26, 28. The first nonwoven fabrics 38 constituting the slip-resistant strip 37 are in contact with and releasably engaged to each other so as to prevent the misalignment of the diaper 11B both at the opposite side portions 33, 35 and the front and back waist regions 26, 28 contacted with each other. Even if an external force is applied to the package 10B, the diapers 11B contained in the space 19 therefore do not lose their folded shape improperly.

The package 10B can maintain the folded shape of the diapers 11B by using the slip-resistant strip 37. Without partial distortion of the film 12 by the diapers 11B, it is possible to prevent the package 10B from losing its shape. Since the package 10B does not lose its shape, a plurality of the packages can be stacked perpendicularly one after another stably and the stacked packages 10B do not collapse improperly.

The releasable engagement strength between the first nonwoven fabrics 38 constituting the slip-resistant strip 37 ranges from 0.5 to 1.5 N/25 mm. The releasable engagement strength between the first nonwoven fabrics 38 less than 0.5 N/25 mm cannot prevent the misalignment of the front and back waist regions 26, 28 of the diaper 11B, and an external force applied to the package 10B easily causes the shape loss of the folded diaper 11B, which may lead to the shape loss of the package 10B. The releasable engagement strength between the first nonwoven fabrics 38 was measured in a similar manner to that employed for the diaper 11A as illustrated in FIGS. 2 to 4.

The dynamic friction coefficient of the first nonwoven fabrics 38 constituting the slip-resistant strip 37 ranges from 0.5 to 1.5. The dynamic friction coefficient of the first nonwoven fabrics 38 less than 0.5 cannot prevent the misalignment of the front and back waist regions 26, 28 of the diaper 11B, and an external force applied to the package 10B easily causes the shape loss of the folded diaper 11B, which may lead to the shape loss of the package 10A. The dynamic friction coefficient of the first nonwoven fabrics 38 was measured in accordance with the method as described in page 3.1 of JIS P 8147. The dynamic friction coefficient was measured in a similar manner to that employed for the diaper 11A as illustrated in FIGS. 2 to 4.

The composite nonwoven fabric constituting the slip-resistant strip 37 has a tensile strength ranging from 30 to 70 N/inch, a basis weight ranging from 30 to 100 g/m², and a thickness ranging from 0.2 to 0.7 mm. The elastic fibers constituting the first nonwoven fabric 38 have a fineness ranging from 0.5 to 20 μm.

In the diapers 11A, 11B, the slip-resistant strip 37 may be attached to at least a part of the opposite side portions 33 extending in the front waist region 26 and at least a part of the opposite side portions 35 extending in the back waist region 28. In the diaper 11A, the slip-resistant strip 36 attached to the front and back end edges 30, 31 can be omitted. The packages 10A, 10B may each contain three or more rows of a plurality of diapers 11A, 11B, respectively. The number of the diapers 11A or 11B forming one row is not limited to that shown in the diagram, and can be made greater or smaller than it. The number of the diapers 11A or 11B constituting one row is preferably 8 or greater.

The bag 12 is made of a plastic film obtained by stretching a thermoplastic synthetic resin. Examples of the synthetic resin include polyester synthetic resins, polyacrylonitrile synthetic resins, polyvinyl chloride synthetic resins, polyethylene synthetic resins, polypropylene synthetic resins and polystyrene synthetic resins.

As the topsheet 22, any one of hydrophilic fiber nonwoven fabrics, hydrophobic fiber nonwoven fabrics having a number of pores, and plastic films having a number of micropores can be used. As the backsheet 23, any one of hydrophobic fiber nonwoven fabrics, air-permeable liquid-impervious plastic films, composite nonwoven fabrics obtained by laminating at least two hydrophobic fiber nonwoven fabrics, and composite sheets obtained by laminating a hydrophobic fiber nonwoven fabric and an air-permeable liquid-impervious plastic film can be used. As the leak-barrier sheet 25, hydrophobic fiber nonwoven fabrics subjected to water repelling treatment can be used. As the backsheet 23 or leak-barrier sheet 25, composite nonwoven fabrics (SM nonwoven fabric, SMS nonwoven fabric, SMMS nonwoven fabric) obtained by superposing a spunbond nonwoven fabric having a high strength and good flexibility on either or both sides of a meltblown nonwoven fabric having high water resistance can be used.

As the nonwoven fabric to be used for the topsheet and backsheet 22, 23 or the leak-barrier sheet 25, nonwoven fabrics produced by any one of the processes such as spunlace, needle punch, meltblown, thermal bond, spunbond and chemical bond. Examples of the fibers constituting the nonwoven fabric include polyester, polyacrylonitrile, polyvinyl chloride, polyethylene, polypropylene and polystyrene fibers. Additional examples include core-sheath composite fibers, side-by-side composite fibers, dual hollow fibers, microporous fibers and junction type composite fibers.

The core 24 is a mixture of a highly absorbent polymer in the form of particles or fibers and a fluff pulp or a mixture of a highly absorbent polymer in the form of particles or fibers, fluff pulp and fibers of a thermoplastic synthetic resin, and it is compacted into a predetermined thickness. The core 24 is covered wholly with tissue paper (not illustrated) in order to prevent it from losing its shape or to prevent the polymer from dropping off.

For superposition between the topsheet and backsheet 22, 23, bonding of the core 24 to the sheets 22, 23, fixing of the leak-barrier sheet 25 to the sheets 22, 23, and securing of the elastic members 43, 46, 49 to the sheets 22, 23, 25, an adhesive or thermal welding means such as heat sealing or sonic sealing can be used. Examples of the adhesive include hot-melt adhesives, acrylic adhesives and rubber adhesives.

1. A diaper-containing package which comprises an open type disposable diaper having a liquid pervious topsheet, a liquid impervious backsheet and a liquid absorbent core existing therebetween, and a bag made of a flexible film packaging therein a plurality of said diapers, said diapers each having front and back waist regions, a crotch region extending therebetween, front and back end edges extending in the transverse direction, and opposite side portions extending in the longitudinal direction; said bag forming top and bottom walls which are spaced opposite to each other in the perpendicular direction, front and back walls opposite to each other and extending between said top and bottom walls, and opposite side walls opposite to each other and extending between the top and bottom walls; and the plurality of diapers being contained compactly in a space surrounded by said walls with said opposite side portions of each diaper folded inward so as to be sandwiched between said front and back regions and each diaper folded in two inward along said crotch region so as for said topsheet oppose to each other; wherein:
a slip-resistant strip having a higher friction force than that of said topsheet and backsheet is attached to at least a part of the opposite side portions in the front waist region of each diaper and at least a part of the opposite side portions in the back waist region of each diaper; and each of said front and back waist regions is overlapped at the opposite side portions thereof so that said slip-resistant strips attached to said opposite side portions respectively are in contact with and releasably engaged with each other; and
said plurality of diapers are arranged forming a row between the opposite side walls of said bag, said diapers at the waist regions and at the crotch region; and the front and back end edges face to either one of said top and bottom walls while the crotch region faces to the other one of said top and bottom walls.

2. A package according to claim 1, wherein said slip-resistant strip is attached to at least a part of the front end edge extending in the front waist region of said diaper and at least a part of the back end edge extending in the back waist region; and in said folded diaper, said front and back end edges are overlapped each other and the slip-resistant strips attached to the front and back end edges are in contact with and releasably engaged with each other.

3. A package according to claim 1, wherein said slip-resistant strip is made of a composite nonwoven fabric obtained by laminating a first nonwoven fabric made of fibers of a thermoplastic synthetic resin having rubber elasticity with a second nonwoven fabric made of fibers of a polyolefin thermoplastic synthetic resin; and in said slip-resistant strips, the first nonwoven fabrics are in contact with and releasably engaged with each other.

4. A package according to claim 3, wherein said respective first nonwoven fabrics constituting said slip-resistant strips are joined at a releasable engagement strength ranging from 0.5 to 1.5 N/25 mm.

5. A package according to claim 3, wherein said first nonwoven fabric constituting said slip-resistant strip has a dynamic friction coefficient ranging from 0.5 to 1.5.

6. A package according to claim 1, wherein at least two of said rows are arranged perpendicularly in the stacked form; in the rows stacked, the front and back end edges of said diaper face to either one of said top and bottom walls, and the crotch region of said diaper faces to the other one of the top and bottom walls.
